SEQUENCE LISTING (5 sequences)

SEQ ID NO: 1 - protein, 449 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVSFTL

SEQ ID NO: 2 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 3 - protein, 342 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRR

SEQ ID NO: 4 - protein, 421 aa, Homo sapiens
NTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCS

SEQ ID NO: 5 - protein, 421 aa, Homo sapiens
NTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVSFTL

(19) United States
(12) Patent Application Publication
(10) Pub. No.: US 2009/0275046 A1
(43) Pub. Date: Nov. 5, 2009

(54) Complement factor H protein as a biomarker of Parkinson's disease

(75) Inventors: Ira L. Goldknopf, The Woodlands, TX (US); Essam A. Sheta, The Woodlands, TX (US); Jennifer K. Bryson, The Woodlands, TX (US)

Correspondence Address: Benjamin A. Adler, PhD, JD, 8011 Candle Ln., Houston, TX, 77071, US

(73) Assignee: Power3 Medical Products, Inc.

(21) Appl. No.: 11/897,078

(22) Filed: Aug. 29, 2007

(51) Int. Cl. G01N 33/53; C07K 14/00

(52) U.S. Cl. 435/7.1; 530/380

(57) ABSTRACT

The present invention relates to a Complement Factor H protein as a biomarker for neurodegenerative disease, including Parkinson's disease, and the related diseases. More specifically, the present invention relates to the identification of a Complement Factor H protein, useful for the screening, diagnosis, and differentiation between neurodegenerative diseases.

### CROSS REFERENCE TO RELATED APPLICATIONS

This application claims priority to U.S. Utility patent application Ser. No. 11/503,881 filed Aug. 14, 2006 and entitled “Assay for Differentiating Alzheimer's and Alzheimer's-Like Disorders” by inventors Ira L. Goldknopf et al. It also claims priority to U.S. Provisional Patent Application Ser. No. 60/708,992 filed Aug. 17, 2005 and entitled “Assay for Differentiating Alzheimer's and Alzheimer's-Like Disorders” by inventors Ira L. Goldknopf et al. It also claims priority to U.S. Utility patent application Ser. No. 11/507,337 filed Aug. 21, 2006 and entitled “Assay for Diagnosis and Therapeutics Employing Similarities and Differences in Blood Serum Concentrations of 3 forms of Complement C3c and Related Protein Biomarkers between Amyotrophic Lateral Sclerosis and Parkinson's Disease” by inventors Ira L. Goldknopf et al.

### BACKGROUND OF THE INVENTION

1. Field of the Invention

The invention relates to the identification of a biomarker for the detection of neurodegenerative disease. More particularly, the present invention relates to the identification of a Complement Factor H protein as a biomarker useful in the screening, diagnosis, and differential diagnosis of Parkinson's disease (PD), and Parkinson's disease Like (PD-Like) disorders.

2. Description of the Related Art

Proteomics is a new field of medical research wherein proteins are identified and linked to biological functions, including roles in a variety of disease states. With the completion of the mapping of the human genome, the identification of unique gene products, or proteins, has increased exponentially. In addition, molecular diagnostic testing for the presence of certain proteins already known to be involved in certain biological functions has progressed from research applications alone to use in disease screening and diagnosis for clinicians. However, proteomic testing for diagnostic purposes remains in its infancy. There is, however, a great deal of interest in using proteomics for the elucidation of potential disease biomarkers.

Detection of abnormalities in the genome of an individual can reveal the risk or potential risk for individuals to develop a disease. The transition from such risk to the emergence of disease can be characterized as an expression of genomic abnormalities in the proteome. Thus, the appearance of abnormalities in the proteome signals the beginning of the process of cascading effects that can result in the deterioration of the health of the patient. Therefore, detection of proteomic abnormalities at an early stage is desirable in order to allow for detection of disease either before it is established or in its earliest stages where treatment may be most effective.

Recent progress using a novel form of mass spectrometry called surface enhanced laser desorption and ionization time of flight (SELDI-TOF) for the testing of ovarian cancer has led to an increased interest in proteomics as a diagnostic tool (Petrocoin, E. F. et al. 2002. Lancet 359:572-577). Furthermore, proteomics has been applied to the study of breast cancer through use of 2D gel electrophoresis and image analysis to study the development and progression of breast carcinoma in patients (Kuerer, H. M. et al. 2002. Cancer 95:2276-2282). In the case of breast cancer, breast ductal fluid specimens were used to identify distinct protein expression patterns in bilateral matched pair ductal fluid samples of women with unilateral invasive breast carcinoma.

Detection of biomarkers is an active field of research. For example, U.S. Pat. No. 5,958,785 discloses a biomarker for detecting long-term or chronic alcohol consumption. The biomarker disclosed is a single biomarker and is identified as an alcohol-specific ethanol glycoconjugate. U.S. Pat. No. 6,124,108 discloses a biomarker for mustard chemical injury. The biomarker is a specific protein band detected through gel electrophoresis and the patent describes use of the biomarker to produce protective antibodies or in a kit to identify the presence or absence of the biomarker in individuals who may have been exposed to mustard poisoning. U.S. Pat. No. 6,326,209 discloses measurement of total urinary 17 ketosteroid-sulfates as biomarkers of biological age. U.S. Pat. No. 6,693,177 discloses a process for preparation of a single biomarker specific for 0-2 acetylated sialic acid and useful for diagnosis and outcome monitoring in patients with lymphoblastic leukemia.

Neurodegenerative diseases such as Parkinson's disease are difficult to diagnose, particularly in their earlier stages. Currently there are no biomarkers available for either early diagnosis or use as drug targets for treatment of neurodegenerative diseases such as Parkinson's (PD) disease.

Neurodegenerative diseases are a varied assortment of central nervous system disorders characterized by the progressive loss of neural tissues. The estimated cost to society of three of these devastating diseases exceeds $100 billion (Sheta E. A. et al. 2006, Expert Rev. Proteomics 3: 45-62). Generally, the diagnosis of Alzheimer's disease, Parkinson's disease or ALS is based on clinical criteria and the results of electro diagnostic studies. Numerous neurological imaging, blood, and CSF studies may be performed, mostly to rule out the presence of other medical conditions that may mimic the clinical appearance of the three diseases. The genetic based diagnostics for Alzheimer's disease, PD, and ALS are associated with the less common familial forms of the diseases, while minimal diagnostics are available for the more common sporadic forms of the diseases. Available protein based diagnostic tests are limited to cerebrospinal fluid, using commercially available immunoassay kits for Amyloid β, Tau protein and phosphorylated Tau, as risk factors for Alzheimer's disease. Absolute dependence on individual markers for diagnosis is problematical as concentrations vary among individuals according to their age, sex and genetic profile. In fact, in AD the only objective definitive diagnostics requires tissue examination, which is usually delayed until autopsy.

Parkinson's disease (PD) is the second most common neurodegenerative disease in adults, with estimated life span of 10 years from the diagnosis of the disease (Sheta E. A. et al. 2006, Expert Rev. Proteomics 3: 45-62). It results primarily from the death of dopaminergic neurons in the substantia nigra. The loss of dopamine production from these cells results in the primary symptoms of PD, which includes slowness of movements, muscle stiffness, tremor, rigidity, and difficulty with balance (Le W and Appel S H 2004, Cur Opin Pharmacology. 4: 79-84). Familial early-onset parkinsonism is linked to an increasing number of different gene mutations, including α-Synuclein (PARK1 (Polymeropoulos M H, et al. 1997 Science. 276: 2045-2047), Parkin (PARK2) (Kitada T, et al. 1998 Nature 392: 605-608), DJ-1 (PARK7) (Bonifati V et al. 2003 Science 299: 256-259), PTEN-induced putative kinase I (PARK6) (Valente E M et al. 2004 Ann Neurol. 56: 336-341), and Leucine-rich repeat kinase 2 (PARK8) (Zimprich A, et al. 2004 Neuron. 44: 601-607). The aggregated proteins form Lewy Bodies (LB), which are a major characteristic of the disease. The major component of Lewy bodies is α-Synuclein. However, individuals with inherited PD linked to Parkin mutation are lacking Lewy bodies. Familial late-onset PD is linked to mutations in PARK8 (Goldwurm S et al. 2005 J Med Genet. 42: e65; Infante J, et al. 2005 Neurosci Lett. In press) and to α-Synuclein (Farrer M, et al. 2001 Hum Mol Genet. 10: 1847-1851). The link of Apo E to PD has also been studied with inconclusive results. Some authors reported a significant risk effect of ApoE4 allele (Martinez M, et al. 2005 Am J Med Genet B Neuropsychiatric Genet. 136: 72-74) while others linked the risk to ApoE2 allele (Huang X et al. 2004 Neurology. 62: 2198-2202).

Sporadic PD is characterized by α-Synuclein inclusions, the distribution of such inclusions relating to disease progression and severity (Muller C M, et al. 2005 J Neuropathol Exp Neurol. 64: 623-628). A study using Affymetrix oligonucleotide micro arrays in the substantia nigra of sporadic PD revealed down regulation of genes related to signal transduction, protein degradation (e.g., ubiquitin-proteasome subunits), dopaminergic transmission/metabolism, iron transport, protein modification/phosphorylation, and energy pathways/glycolysis functional classes (Mandel S et al. 2005 Ann N Y Acad Sci. 1053: 356-375). A major finding is the decreased expressions of 5 subunits of the UPS, SKP1A, a member of the SCF (E3) ubiquitin ligase complex, and chaperone HSC-70. In addition, the authors observed up regulation of genes related to cell adhesion/cytoskeleton, extra cellular matrix components, cell cycle, protein modification/phosphorylation, protein metabolism and transcription, and inflammation/hypoxia (e.g., key iron and oxygen sensor EGLN1) classes.

Studies indicate that general physicians make an incorrect initial diagnosis of PD in between 8% and 35% of cases. Even general neurologists have some difficulties in correctly identifying the disease. Ongoing studies are attempting to identify ways of slowing the deterioration of the dopamine-producing cells (Samii A, et al. 2004 Lancet 363: 1783-1793). It is believed that the symptoms of PD do not appear until about 80% of the dopamine-producing cells are already dead or impaired (Quinn N P 1997 Baillieres Clin Neurol. 6, 1-13). Therefore, earlier identification of those with PD may help to optimize the management of these patients, especially if methods of neuroprotection are developed.

Therefore, there remains a need for better ways to detect, diagnose, and distinguish PD from other neurodegenerative diseases, including a need for specific biomarkers of Parkinson's disease that distinguish patients with Parkinson's disease from patients with Parkinson's disease Like disorders with similar symptoms to Parkinson's disease.

### SUMMARY OF THE INVENTION

The present invention relates to a Complement Factor H protein as a biomarker for neurodegenerative disease, whereby the concentration of Complement Factor H protein in the serum of Parkinson's disease patients is significantly higher than age-matched control subjects. In addition, the concentrations of a Complement Factor H protein in the serum of patients with Frontotemporal dementia (FTD), Lewy body dementia (LBD), Corticalbasal Ganglionic Degeneration (CBGD), or with a form of Multiple System Atrophy (MSA I), are significantly lower than normal controls or patients with Parkinson's disease, and also significantly lower than patients with Stroke-Related, Mixed, or other PD-Like disorders, including another form of Multiple System Atrophy (MSA II).

One aspect of the present invention is the use of the biomarker, a Complement Factor H protein, for screening, diagnosis, or differential diagnosis of Parkinson's disease comprising: obtaining a blood serum sample from a test subject; determining the quantity of a Complement Factor H protein in the blood serum sample; and determining the ranges of the quantity of a Complement Factor H protein in blood serum samples from normal control individuals, from patients with Parkinson's disease, from patients with other neurological disorders including Lewy body dementia, Frontotemporal dementia, Corticalbasal Ganglionic Degeneration, Multiple System Atrophy, other non-Parkinson's neurological disorders, and Mixed neurological disorders, whereby the quantity of a Complement Factor H protein in the blood serum sample of the test subject within the range of Parkinson's disease values is indicative of the presence of Parkinson's disease, and the quantity of a Complement Factor H protein in the blood serum sample of the test subject outside the range of Parkinson's disease values is indicative of the absence of Parkinson's disease and the presence of a normal condition or a Parkinson's Like disease such as Lewy body dementia, Frontotemporal dementia, Corticalbasal Ganglionic Degeneration, or a form of Multiple System Atrophy.

Yet another aspect of the present invention is the use of the biomarker, a Complement Factor H protein, for screening, diagnosis, or differential diagnosis of Parkinson's disease like disorders, comprising: obtaining a blood serum sample from a test subject; determining the quantity of a Complement Factor H protein in the blood serum sample; and determining the ranges of the quantity of a Complement Factor H protein in blood serum samples from normal control individuals, from patients with Parkinson's disease-like neurological disorders including Lewy body dementia, Frontotemporal dementia, Corticalbasal Ganglionic Degeneration, Multiple System Atrophy, Stroke-related, Mixed and other non-Parkinson's neurological disorders, whereby the quantity of a Complement Factor H protein in the blood serum sample of the test subject within the range of values of Lewy body dementia, Frontotemporal dementia, Corticalbasal Ganglionic Degeneration, a form of Multiple System Atrophy, is indicative of the presence of Lewy body dementia, Frontotemporal dementia, Corticalbasal Ganglionic Degeneration, or a form of Multiple System Atrophy, and the quantity of a Complement Factor H protein in the blood serum sample of the test subject outside the range of values of Lewy body dementia, Frontotemporal dementia, Corticalbasal Ganglionic Degeneration, a form of Multiple System Atrophy, is indicative of the presence of a normal condition, Parkinson's disease, or a Stroke-related, Mixed, or other non-Parkinson's neurological disorder, including another form of Multiple System Atrophy.

Yet another aspect of the present invention is the use of the biomarker, a Complement Factor H protein, for differential diagnosis, or for screening of Parkinson's disease, comprising: obtaining a blood serum sample from a test subject; determining the quantity of a Complement Factor H protein in the blood serum sample; and determining the ranges the quantity of a Complement Factor H protein in blood serum samples from normal control individuals, from patients with Parkinson's disease, and patients with other neurological disorders including Lewy body dementia, Frontotemporal dementia, Multiple System Atrophy, Corticalbasal Ganglionic Degeneration, Other non-Parkinson's neurological disorders, and Mixed neurological disorders, by two-dimensional gel electrophoresis; quantitating a Complement Factor H protein in the protein expression pattern; whereby the quantity of a Complement Factor H protein in the blood serum sample of the test subject within the range of Parkinson's disease values is indicative of the presence of Parkinson's disease, and the quantity of a Complement Factor H protein in the blood serum sample of the test subject outside the range of Parkinson's disease values is indicative of the absence of Parkinson's disease, the presence of a normal condition or a non-Parkinson's neurological disorder such as Lewy body dementia, Frontotemporal dementia, or a form of Multiple System Atrophy.

Yet another aspect of the present invention is the use of the biomarker, a Complement Factor H protein, for screening, diagnosis, or differential diagnosis of Parkinson's disease like disorders, comprising: obtaining a blood serum sample from a test subject; determining the quantity of a Complement Factor H protein in the blood serum sample; and determining the ranges of the quantity of a Complement Factor H protein in blood serum samples from normal control individuals, from patients with Parkinson's disease-like neurological disorders including Lewy body dementia, Frontotemporal dementia, Corticalbasal Ganglionic Degeneration, Multiple System Atrophy, Stroke-related, Mixed and other non-Parkinson's neurological disorders, by two-dimensional gel electrophoresis; quantitating a Complement Factor H protein in the protein expression pattern; whereby the quantity of a Complement Factor H protein in the blood serum sample of the test subject within the range of values of Lewy body dementia, Frontotemporal dementia, Corticalbasal Ganglionic Degeneration, a form of Multiple System Atrophy, is indicative of the presence of Lewy body dementia, Frontotemporal dementia, Corticalbasal Ganglionic Degeneration, or a form of Multiple System Atrophy, and the quantity of a Complement Factor H protein in the blood serum sample of the test subject outside the range of values of Lewy body dementia, Frontotemporal dementia, Corticalbasal Ganglionic Degeneration, a form of Multiple System Atrophy, is indicative of the presence of a normal condition, Parkinson's disease, or a Stroke-related, Mixed, or other non-Parkinson's neurological disorder, including another form of Multiple System Atrophy.

Yet another aspect of the present invention is the use of the biomarker, a Complement Factor H protein, for differential diagnosis, or for screening of a neurodegenerative disease, comprising: obtaining a patient blood serum sample; determining the quantity of a Complement Factor H protein by an immunoassay using an antibody that recognizes a Complement Factor H protein; whereby the quantity of a Complement Factor H protein in the blood serum sample of the test subject within the range of Parkinson's disease values is indicative of the presence of Parkinson's disease, and the quantity of a Complement Factor H protein in the blood serum sample of the test subject outside the range of Parkinson's disease values is indicative of the absence of Parkinson's disease, the presence of a normal condition, or a non-Parkinson's disorder such as Lewy body dementia, Frontotemporal dementia, Corticalbasal Ganglionic Degeneration, or a form of Multiple System Atrophy.

Yet another aspect of the present invention is the use of the biomarker, a Complement Factor H protein, for differential diagnosis, or for screening of a neurodegenerative disease, comprising: obtaining a patient blood serum sample; determining the quantity of a Complement Factor H protein by an immunoassay using an antibody that recognizes a Complement Factor H protein; whereby the quantity of a Complement Factor H protein in the blood serum sample of the test subject within the range of values of Lewy body dementia, Frontotemporal dementia, Corticalbasal Ganglionic Degeneration, or a form of Multiple System Atrophy, is indicative of the presence of Lewy body dementia, Frontotemporal dementia, Corticalbasal Ganglionic Degeneration, or a form of Multiple System Atrophy, and the quantity of a Complement Factor H protein in the blood serum sample of the test subject outside the range of values of Lewy body dementia, Frontotemporal dementia, Corticalbasal Ganglionic Degeneration, or a form of Multiple System Atrophy, is indicative of the presence of a normal condition, Parkinson's disease, or a Stroke-related, Mixed, or other non-Parkinson's neurological disorder, including another form of Multiple System Atrophy.

The foregoing has outlined rather broadly several aspects of the present invention in order that the detailed description of the invention that follows may be better understood. Additional features and advantages of the invention will be described hereinafter which form the subject of the claims of the invention. It should be appreciated by those skilled in the art that the conception and the specific embodiment disclosed might be readily utilized as a basis for modifying or redesigning the structures for carrying out the same purposes as the invention. It should be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 illustrates the differentially expressed proteins detected in a 2D gel of blood serum collected from a patient: a) where the spot intensities indicative of the concentration of the proteins in blood serum cover a dynamic range of Four orders of magnitude; and b) where the indicated protein (spot N4411) is estimated to have Mwt 39 KD pI 6.3, and is identified by LC-MS/MS of tryptic peptides as a Complement Factor H processing product protein.

FIG. 2 is a comparative statistical Box and Whiskers graph (constructed using Analyze-it software for Microsoft Excel), illustrating the differential expression level of a Complement Factor H processing product protein (spot N4411) in blood serum, based on the data from:
57 normal control individuals (Controls),
29 Parkinson's disease patients (PD),
8 patients with certain Parkinson's Disease-Like disorders, including:
   Frontotemporal dementia (FTD, 2 patients),
   Lewy body dementia (LBD, 2 patients), and
   A form of Multiple System Atrophy (MSA I, 3 patients)
   Corticalbasal Ganglionic Degeneration (CBGD, 1 patient)
19 patients with Stroke-Related, Mixed and other PD-Like disorders, including patients with:
Stroke Related disorders (5 patients), including:
   Multi-infarct dementia (1 patient),
   Cerebrovascular Accident (Stroke, CVA, 1 patient),
   Post-irradiation Encephalopathy, Seizures (1 Patient),
   Vascular Parkinsonism (1 patient),
   Thalamic CVA, history (fix of Lung Cancer (CA) (1 patient),
Mixed disorders (9 patients), including:
   Alzheimer's disease combined with Vascular (Multi-Infarct) dementia (1 patient),
   Alzheimer's disease combined with Lewy body dementia (3 patients)
   Parkinson's disease combined with Lewy body dementia (1 patient)
   Alzheimer's and Parkinson's disease combined with Lewy body dementia (1 patient).
   Frontotemporal dementia combined with Parkinsonism (1 patient)
   Frontotemporal dementia combined with Chronic Inflammatory Demyelinating Polyneuropathy (1 patient),
   Multiple System Atrophy combined with a Subdural Hematoma (1 patient),
Other Parkinson's disease-like disorders (5 patients), including:
   Alcohol related dementia (1 patient),
   Semantic dementia (1 patient),
   Idiopathic Sensory Ataxia (1 patient), and
   Another form of Multiple System Atrophy (MSA II, 2 patients).

Also depicted in FIG. 2 are example concentration ranges, based on the data presented in the graph, for the purpose of illustrating preferred embodiments of the invention, including:

The concentration range of 0-165 PPM, where this range would correspond to the serum concentrations of the Complement Factor H processing product protein (spot N4411) of individuals who are normal controls or patients who have Lewy body dementia, Frontotemporal dementia, Corticalbasal Ganglionic Degeneration, or a form of Multiple System Atrophy (MSA I).

The concentration range of ≧166 PPM, where this range would correspond to the serum concentrations of the Complement Factor H processing product protein (spot N4411) of patients who have Parkinson's disease or have a Stroke-Related, Mixed, MSA II, or another individual PD-Like disorder.

FIG. 3 is a plot of the Receiver Operator Characteristics of blood serum concentrations of the Complement Factor H processing product protein (spot N4411) in distinguishing between patients with Parkinson's disease (PD) and patients with PD-Like disorders, including Lewy Body Dementia (LBD), Frontotemporal dementia (FTD), Cortical Basal Ganglionic Degeneration (CBGD), and a form of Multiple System Atrophy (MSA I).

FIG. 4 is a plot of the Receiver Operator Characteristics of blood serum concentrations of the Complement Factor H processing product protein (spot N4411) in distinguishing between patients with PD-Like disorders, including Lewy Body Dementia (LBD), Frontotemporal dementia (FTD), Cortical Basal Ganglionic Degeneration (CBGD) and a form of Multiple System Atrophy (MSA I) vs. patients with Stroke-related, Mixed, or other non-Parkinson's neurological disorders, including another form of Multiple System Atrophy.

FIG. 5 is a plot of the Receiver Operator Characteristics of blood serum concentrations of the Complement Factor H processing product protein (spot N4411) in distinguishing between patients with Parkinson's disease (PD) and Normal Controls.

FIG. 6 is a plot of the Receiver Operator Characteristics of blood serum concentrations of the Complement Factor H processing product protein (spot N4411) in distinguishing between Normal Controls and patients with Stroke-related, Mixed, or other non-Parkinson's neurological disorders, including another form of Multiple System Atrophy.

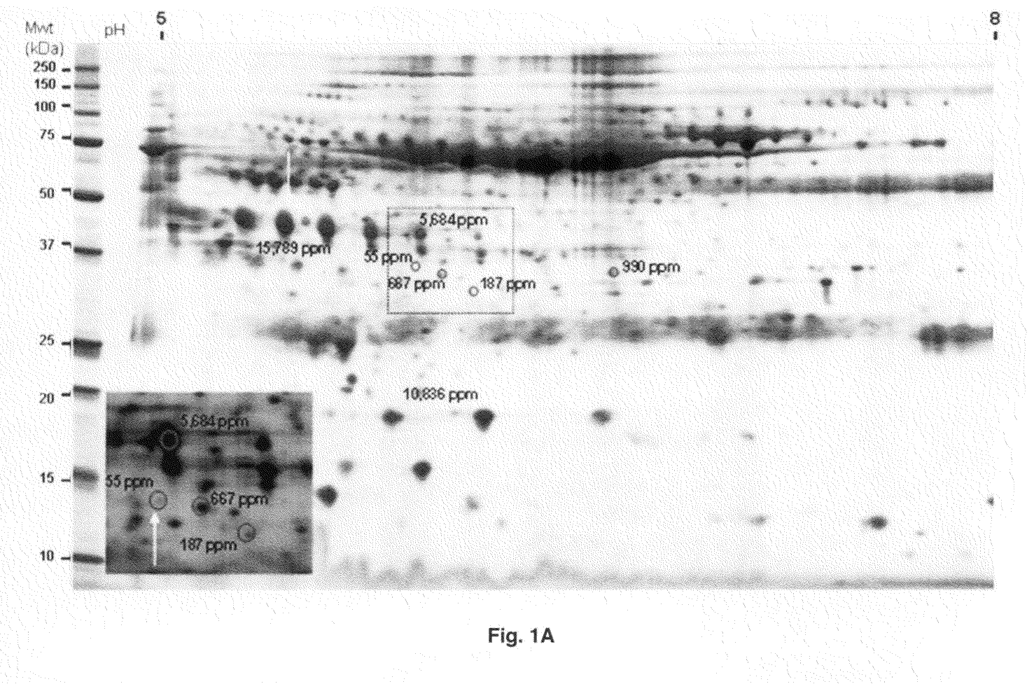

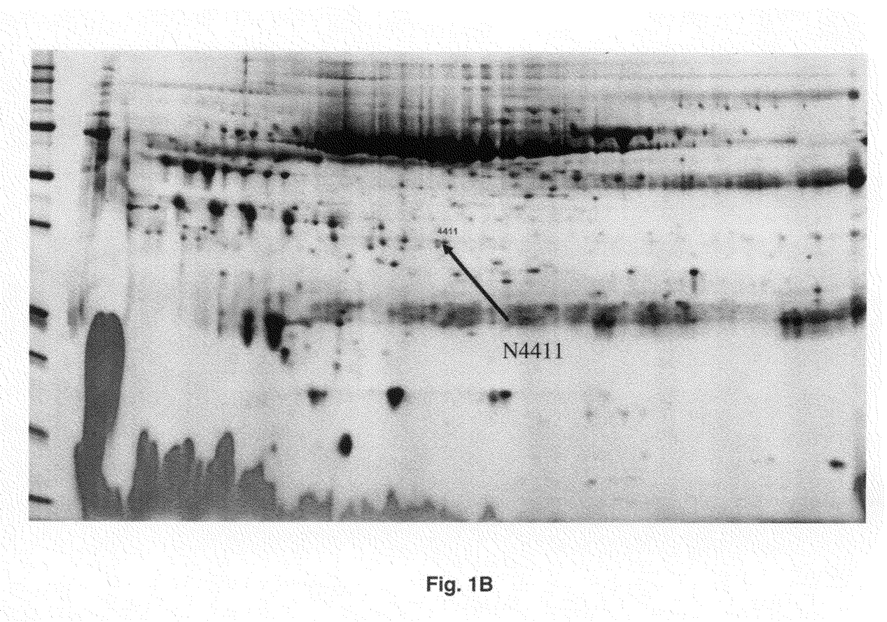

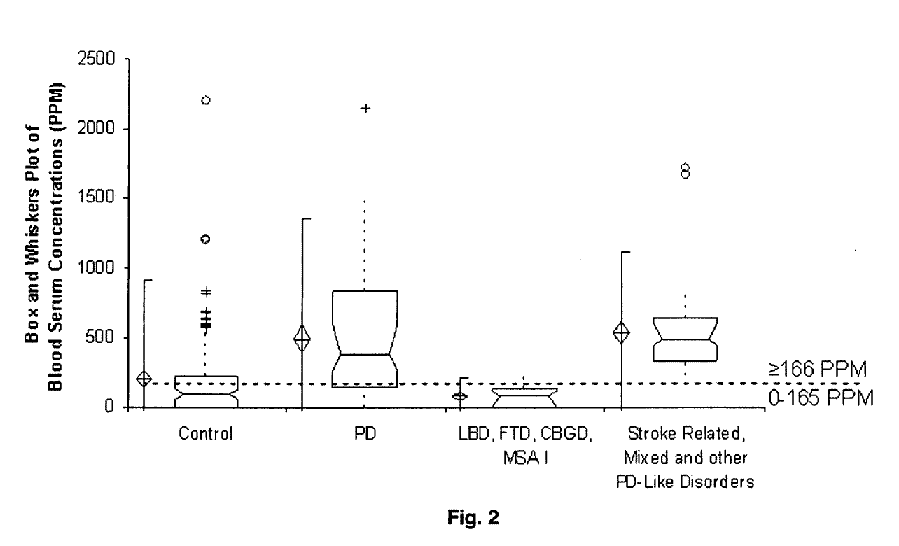

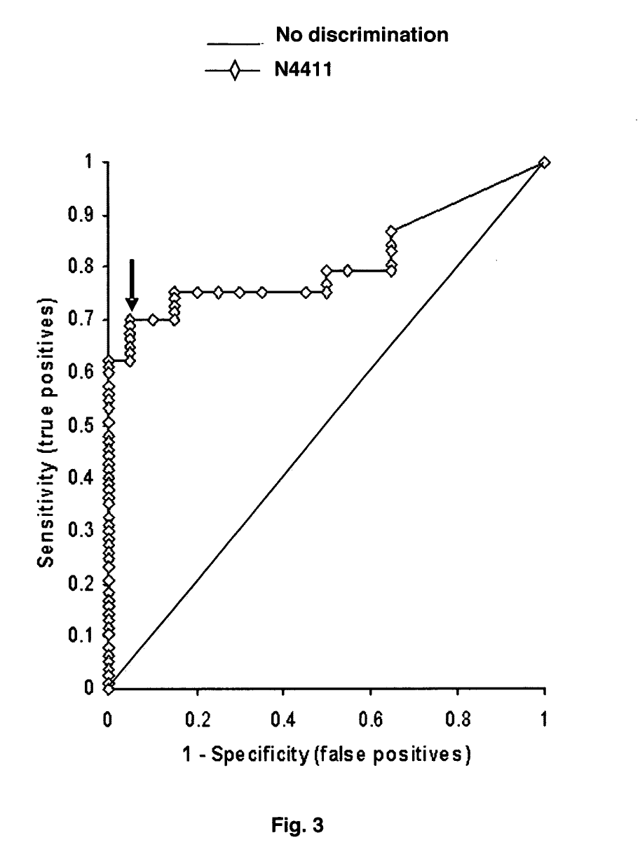

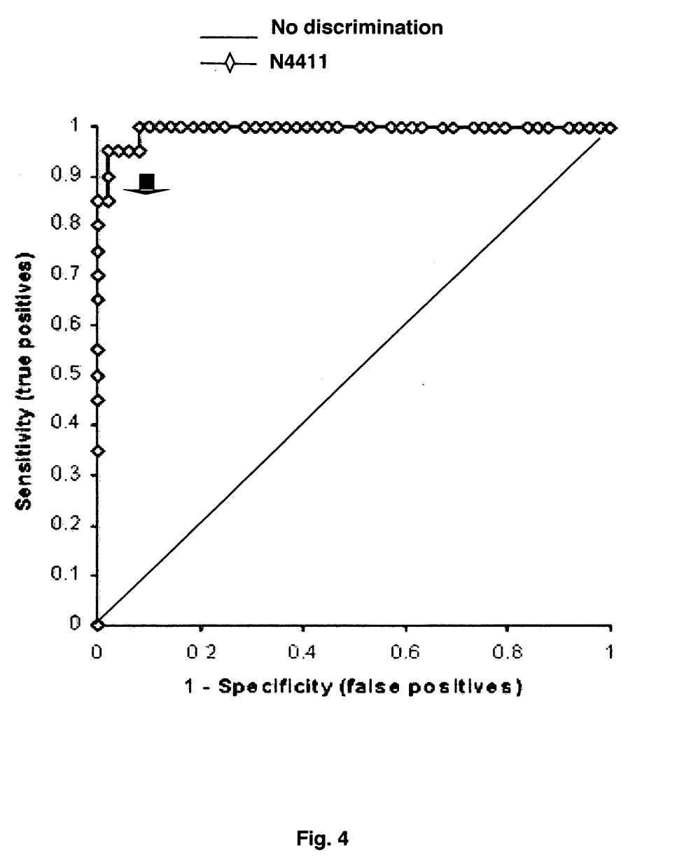

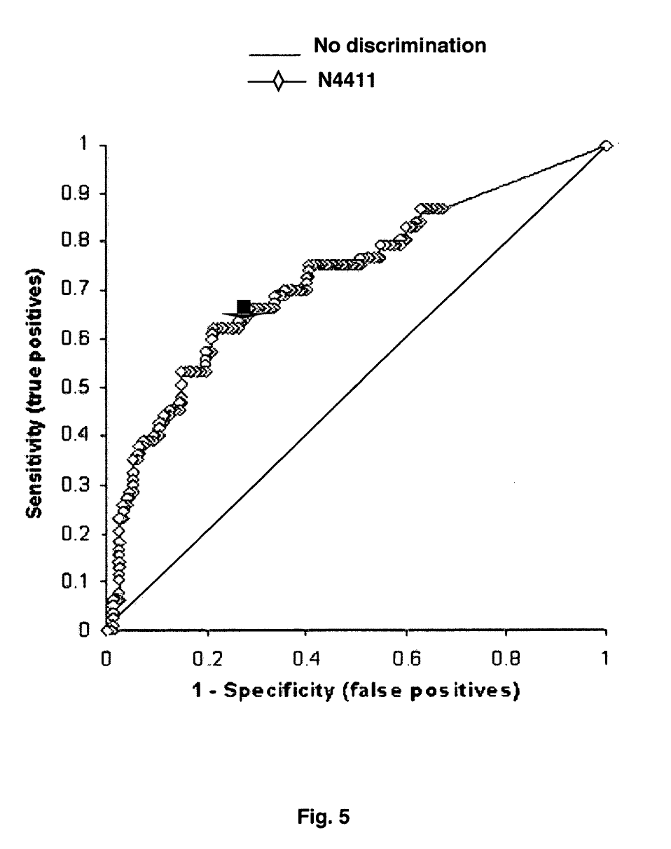

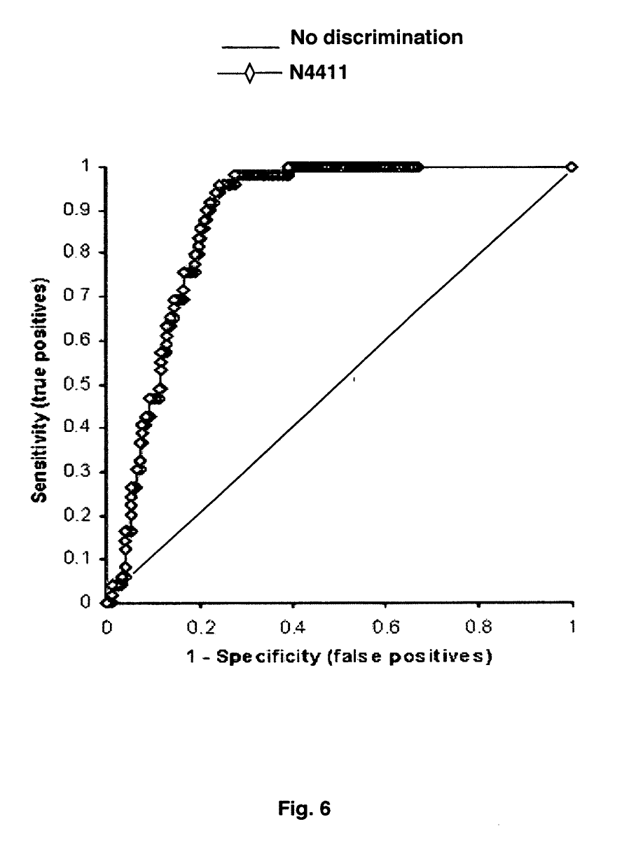

Table 1 depicts the reproducibility of quantitation in 2D gels. Table 1a wherein 9 replicate analyses were performed with an individual sample of bovine serum albumin, where the sample was separated by 2D gel electrophoresis into a characteristic set of 5 spots which were then subjected to quantitation. The raw density counts (Gaussian Peak Values) are shown as are the individual values, averages, standard deviations, % Coefficients of Variation, and mass of the protein in nanograms (ng) for each spot. Table 1b illustrates the reproducibility of quantitation of protein spots over the dynamic range of the 2D gel assay of human serum depicted in FIG. 1a. Shown are replicate (14×) 2D gel analyses each of the quantitation of 13 different protein spots ranging from 13,542 ppm to 72 ppm with a coefficient of variation of ≦20%, where 72 ppm is approximately 10 fold higher than the limit of detection (LOD=5-10 ppm) of the assay.

Table 2 illustrates the identification of the amino acid sequence of protein spot N4411 as a processing product derived from Complement Factor H Isoform b (Hs, short splice form, Sequence 1).

Table 3 illustrates the identification of the amino acid sequence of protein spot N4411 best fit as a processing product derived from Complement Factor H Isoform a (long splice form, Sequence 2).

Table 4 illustrates the identification of the amino acid sequence of protein spot N4411 as a processing product derived from Complement Factor H Isoform a (H, long splice form) and/or Complement Factor H Isoform b (Hs, short splice form). Included are (Table 4A) the best fit amino acid sequence (Sequence 3) found in both Isoforms a and b (amino acids 1-342), as well as (Table 4B Sequence 4, 4C sequence 5) the shortest amino acid sequences that would be unique to Isoform a (amino acids 29-449 of isoform a, Sequence 4); or to isoform b, (amino acids 29449 of isoform b, Sequence 4); both of which are too large to fit the measured MW of Complement Factor H processing product protein (spot N4411).

Table 5 depicts (i) the differential expression level and percent expression difference from control of blood serum concentrations of Complement Factor H processing product protein (spot N4411), depicted in FIG. 2, of the groups of 57 normal controls, 29 Parkinson's disease patients (PD), 8 patients with Frontotemporal dementia (FTD), Lewy body dementia (LBD), Corticalbasal Ganglionic Degeneration), or a form of Multiple System Atrophy (MSA I), 19 patients with Stroke-Related, Mixed and other AD/PD-Like disorders, including another form of Multiple System Atrophy (MSA II).

(ii) Statistical significance of compared group mean levels (ppm), of serum Complement Factor H, using analysis of variance (wherein ANOVA-P≦0.05, as constructed using Analyze-it software for Microsoft Excel, is considered to be statistically significant).

### DESCRIPTION OF THE PREFERRED EMBODIMENTS

The present invention relates to a Complement Factor H protein as a biomarker for Parkinson's disease. More particularly, the present invention relates to the identification of a Complement Factor H protein as a biomarker useful for the detection, diagnosis, and differentiation of patients with Parkinson's disease, from normal individuals and patients with other neurological disorders that are not Parkinson's disease, including Lewy body dementia, Frontotemporal dementia, Corticalbasal Ganglionic Degeneration, and a form of Multiple system atrophy (MSA I). The present invention also relates to the identification of a Complement Factor H protein as a biomarker useful for the detection, diagnosis, and differentiation of normal controls and patients with Lewy body dementia, Frontotemporal dementia, Corticalbasal Ganglionic Degeneration, and a form of Multiple system atrophy (MSA I) from patients with Stroke-related, Mixed, or other non-Parkinson's neurological disorders, including another form of Multiple System Atrophy (MSA II)

The method for identification of a Complement Factor H protein as a biomarker for neurodegenerative disease is based on the comparison of 2D gel electrophoretic images of serum obtained from normal human control subjects and human subjects with and without diagnosed Parkinson's disease, Lewy body dementia, Frontotemporal dementia, Corticalbasal Ganglionic Degeneration, and a form of Multiple system atrophy (MSA I), Stroke-related, Mixed, or other non-Parkinson's neurological disorders, including another form of Multiple System Atrophy (MSA II) 2D gel electrophoresis has been used in research laboratories for biomarker discovery since the 1970's (Margolis J. et al. 1969, Nature. 1969 221: 1056-1057; Orrick, L. R. et al. 1973; Proc Nat'l Acad Sci. USA. 70: 1316-1320; Goldknopf, I. L. et al. 1975, J Biol Chem. 250: 7182-7187; Goldknopf, I. L. et al. 1977, Proc Nat'l Acad Sci USA. 74: 5492-5495; O'Farrell, P. H. 1975, J. Biol. Chem. 250: 4007-4021; Anderson, L. 1977, Proc Nat'l Aced Sci USA. 74: 864-868; Klose, J. 1975, Human Genetic. 26: 231-243). In the past, this method has been considered highly specialized, labor intensive and non-reproducible. Only recently with the advent of integrated supplies, robotics, and software, combined with bioinformatics, has progression of this proteomics technique in the direction of diagnostics become feasible. The promise and utility of 2D gel electrophoresis is based on its ability to detect changes in protein expression and to discriminate protein isoforms that arise due to variations in amino acid sequence and/or post-synthetic protein modifications such as phosphorylation, ubiquitination, conjugation with ubiquitin-like proteins, acetylation, glycosylation, and proteolytic processing. These are important variables in cell regulatory processes that are differentially expressed in blood serum biomarkers in neurodegenerative diseases, including Alzheimer's and Parkinson's diseases, and ALS (Goldknopf, I. L. et al. U.S. Utility patent application Ser. No. 11/507,337, Goldknopf I. L. et al. 2006 Biochem. Biophys. Res. Commun. 342: 1034-1039; Sheta E. A. et al. 2006, Expert Rev. Proteomics 3: 45-62; Goldknopf I. L 2007, Expert Review of Molecular Diagnostics 7: 339-343.).

There are few comparable alternatives to 2DGE for tracking changes in protein expression patterns related to disease. The introduction of high sensitivity fluorescent staining, digital image processing and computerized image analysis has greatly amplified and simplified the detection of unique species and the quantification of proteins. By using known protein standards as landmarks within each gel run, computerized analysis can detect unique differences in protein expression and modifications between two samples from the same individual or between several individuals.

Proteins of interest can be excised from the gels and the proteins can then be identified by in-gel digestion and matrix assisted laser desorption time of flight mass spectroscopy (MALDI-TOF MS) based peptide mass fingerprinting and database searching, or liquid chromatography with tandem mass spectrometry partial sequencing of individual peptides (LCMS/MS).

The identification of a Complement Factor H protein as a biomarker of neurodegenerative disease was based on a comparison of the 2D gel electrophoretic images of serum samples obtained from 57 normal controls, 29 Parkinson's disease patients (PD), 8 patients with Frontotemporal dementia (FTD), Lewy body dementia (LBD), Corticalbasal Ganglionic Degeneration, and a form of Multiple System Atrophy (MSA I), 19 patients with Stroke-Related, Mixed, and other PD-Like disorders, including another form of Multiple System Atrophy.

### Sample Collection and Preparation

Sample collection and storage have been performed in many different ways depending on the type of sample and the conditions of the collection process. In the present study, blood serum samples were collected from patients, aliquoted and stored in a −80° C. freezer before analysis format.

In a preferred embodiment of the invention, the serum samples were removed from 80° C. and placed on ice for thawing. To each 100 μL of sample, 100 μL of LB-2 buffer (7M urea. 2M Thiourea, 1%/DTT, 1% Triton X-100, 1× Protease inhibitors, and 0.5% Ampholyte pH 3-10) was added and the mixture vortexed. The sample was incubated at room temperature for about 5 minutes.

### Two Dimensional Gel Electrophoresis of Serum Samples

Separation of the proteins in the serum samples was then performed using 2D gel electrophoresis. The 2D gel electrophoretic images were obtained, compared and analyzed as described in the U.S. Provisional Patent Application Ser. No. 60/614,315 entitled “Differential Protein Expression Patterns Related to Disease States” filed Sep. 29, 2004 and incorporated herein by reference. A protein assay was performed on the sample to determine total protein content in μg.

Approximately 100 μg of the solubilized protein pellet was suspended in a total volume of 184 μL of IEF loading buffer containing 1 μL Bromophenol Blue as a marker to trace the progress of the electrophoresis. Each sample was loaded onto an 11 cm IEF strip (Bio-Rad), pH 5-8, and overlaid with 1.5-3.0 ml of mineral oil to minimize the sample buffer evaporation. Using the PROTEAN® IEF Cell, an active rehydration was performed at 50V and 20° C. for 12-18 hours.

IEF strips were then transferred to a new tray and focused for 20 min. at 250V followed by a linear voltage increase to 8000V over 2.5 hours. A final rapid focusing was performed at 8000V until 20,000 volt-hours were achieved. Running the IEF strip at 500V until the strips were removed finished the isoelectric focusing process.

Isoelectric focused strips were incubated on an orbital shaker for 15 min with equilibration buffer (2.5 ml buffer/strip). The equilibration buffer contained 6M urea, 2% SDS, 0.375M HC1, and 20% glycerol, as well as freshly added DTT to a final concentration of 30 mg/ml. An additional 15 min incubation of the IEF strips in the equilibration buffer was performed as before, except freshly added iodoacetamide (C2H4INO) was added to a final concentration of 40 mg/ml. The IPG strips were then removed from the tray using clean forceps and washed five times in a graduated cylinder containing the Bio Rad running buffer 1× Tris-Glycine-SDS.

The washed IEF strips were then laid on the surface of Bio Rad pre-cast CRITERION SDS-gels 8-16%. The IEF strips were fixed in place on the gels by applying a low melting agarose. A second dimensional separation was applied at 200V for about one hour. After electrophoresis, the gels were carefully removed and placed in a clean tray and washed twice for 20 minutes in 100 ml of pre-staining solution containing 10% methanol and 7% acetic acid.

### Staining and Analysis of the 2D Gels

The gels were stained with SYPRO RUBY (Bio-Rad Laboratories) and subjected to fluorescent digital image analysis. The protein patterns of the serum samples were analyzed using PDQUEST™ (Bio-Rad Laboratories) image analysis software.

The 2D gel patterns of the 57 serum samples collected from normal control subjects were compared with each other pursuant to the methodology described in the U.S. Utility patent application Ser. No. 11/172,219 entitled “Differential Protein Expression Patterns Related to Disease States” filed Sep. 29, 2004 and incorporated herein by reference. The 57 normal individual blood serum samples all gave similar 2D gel protein patterns.

These normal protein expression patterns were then compared to the gel patterns obtained with blood serum samples from the 29 Parkinson's disease patients (PD), 8 patients with Frontotemporal dementia (FTD), Lewy body dementia (LBD), Corticalbasal Ganglionic Degeneration (CBGD), a form of Multiple System Atrophy (MSA I), and 19 patients with Stroke-Related, Mixed and other PD-Like disorders, including another form of Multiple System Atrophy (MSA II). When the gel patterns of PD patients were compared to the gel patterns of normal subjects, protein spot 4411, of particular interest, was identified as shown in FIG. 1a. Protein spot 4411 was selected for further investigation. Protein spot 4411 was quantitated by stain intensity in each of the normal and disease patient groups of serum samples.

In order to assess the reproducibility of the 2D gels and staining, 75 nanograms of bovine serum albumin (BSA) was run on 9 separate 2D gels. The gels were stained with SYPRO RUBY and the 5 spots resolved in the BSA region of the gel were then subjected to quantitative analysis using PDQUEST™ and the Gaussian Peak Value method. The results shown in Table 1a illustrate that the electrophoretic patterns were reproducible and the reproducibility (% Coefficient of Variation=% CV) was independent of the spot amount over the range tested (2.9-38.6 ng/spot).

TABLE 1a

| | Spot # | | | | |
|---|---|---|---|---|---|
| Replicate # | 9901 | 9902 | 9904 | 9905 | 9906 |
| 1 | 332 | 1152 | 2612 | 739 | 229 |
| 2 | 246 | 974 | 2694 | 513 | 167 |
| 3 | 336 | 1065 | 2354 | 668 | 225 |
| 4 | 311 | 1272 | 3482 | 713 | 198 |
| 5 | 351 | 1168 | 2724 | 733 | 245 |
| 6 | 268 | 1059 | 2753 | 622 | 184 |
| 7 | 452 | 1630 | 4000 | 946 | 281 |
| 8 | 405 | 1195 | 2752 | 870 | 274 |
| 9 | 258 | 1050 | 2716 | 699 | 189 |
| AVG | 329 | 1174 | 2899 | 723 | 221 |
| STDEV | 68 | 193 | 510 | 127 | 40 |
| % CV | 21% | 16% | 18% | 18% | 18% |
| ng/spot | 4.4 | 15.6 | 38.6 | 9.6 | 2.9 |

Reproducibility of Quantitation in 9 Gels
PDQuest Gaussian Peak Value of the Major Components of BSA

As shown in FIG. 1b, 2D gel electrophoresis of human blood serum, fluorescent staining with SyproRuby, and digital imaging provides a broad dynamic concentration range of protein spots, which are illustrated by the indicated spots with concentrations ranging from a low of 55 ppm spot density to a high of 15,709 ppm spot density (white arrows). Table 1b illustrates the reproducibility of quantitation of 13 different spots with decreasing concentrations over the full dynamic range of the assay, illustrated with protein spots ranging in spot density from a low of 72 ppm to a high of 13,542 ppm. The coefficients of variation were ≦20%, for replicates of 14 gels run on different days with different technicians, and were independent of the concentrations of the protein spots within that range. The limit of detection (LOD) is at a 10 fold lower blood serum concentration than the bottom of that range, or 100 pg/spot ˜5-10 PPM

TABLE 1b

| | | | | | Coefficient | |
|---|---|---|---|---|---|---|
| | | | | Std | of | |
| Biomarker | N | Mean | +/− | Error | Variation | ≦20% |
| M1 | 14 | 13542 | | 711 | 20 | \| |
| M2 | 14 | 3853 | | 140 | 14 | \| |
| M3 | 14 | 1413 | | 52 | 14 | \| |
| M4 | 14 | 1015 | | 49 | 18 | \| |
| M5 | 14 | 678 | | 28 | 15 | \| |
| M6 | 14 | 655 | | 33 | 19 | \| |
| M7 | 14 | 595 | | 31 | 19 | \| |
| M8 | 14 | 469 | | 26 | 20 | \| |
| M9 | 14 | 359 | | 16 | 17 | \| |
| M10 | 14 | 209 | | 11 | 20 | \| |
| M11 | 14 | 129 | | 5 | 15 | \| |
| M12 | 14 | 106 | | 6 | 20 | \| |
| M13 | 14 | 72 | | 4 | 19 | ↓ |

LOD = 100 pg/spot = ~5-10 ppm

### The Isolation and Identification of the Protein Spot 2307

Protein spot N4411 was carefully excised, in-gel digested with trypsin, and tryptic peptides subjected to mass fingerprinting/sequence analysis by high performance liquid chromatography/tandem mass spectrometry (LC-MS/MS) and expert database searching.

Tandem mass spectrometry provides a powerful means of determining the structure and identity of proteins and peptides. The unknown tryptic peptide is first separated and purified by liquid chromatography and then the effluent from the separation is vaporized by electrospray, separated in a mass spectrometer and then bombarded with high-energy electrons causing it to fragment in a characteristic manner, indicative of its amino acid sequence. The fragments, which are of varying mass and charge, are then passed through a magnetic field and separated according to their mass/charge ratios. The resulting characteristic fragmentation pattern of the unknown peptide is used to identify its amino acid sequence.

A protein can often be unambiguously identified by an LC MS/MS analysis of its constituent peptides (produced by either chemical or enzymatic treatment of the sample).

Following differential expression analysis, protein spot N4411 was carefully excised from the gel for identification. Excised gel spots of protein N4411 were de-stained by washing the gel spots twice in 100 mM NH4HCO3 buffer, followed by soaking the gel spots in 100% acetonitrile for 10 minutes. The acetonitrile was aspirated before adding the trypsin solution.

Typically, a small volume of trypsin solution (approximately 5-15 μg/ml trypsin) is added to the de-stained gel spots and incubated at 3 hours at 37° C. or overnight at 30° C. The digested peptides were extracted, washed, desalted and subjected to liquid chromatography followed by tandem mass spectral analysis to identify protein spot N4411. Those of skill in the art are familiar with mass spectral analysis of digested peptides. The mass spectral analysis was conducted on a Micromass LC QTOF (Waters). Peptide fragmentation patterns were obtained from the tryptic in-gel digests of protein spot N4411 and the patterns were subjected to public database searches using the GenBank and dbEST databases maintained by the National Center for Biotechnology Information (hereinafter referred to as the NCBI database). Those of skill in the art are familiar with searching databases, such as the NCBI database. The NCBI database search results were displayed with the best matched amino acid sequences of the identified peptides and the protein accession of number the protein sequence they were derived from. For protein spot 4411, the protein identified by the NCBI database search was a Complement Factor H protein (Tables 2 and 3).

Given the results of 2D gel electrophoresis, wherein the protein spot N4411 has a MW of 39 KD, and a pI of 6.3, it is most likely that the protein spot N4411 corresponds to the Complement Factor H Isoform a and/or b processing product depicted in Table 4a.

### Statistical Analysis:

Receiver Operating Characteristics (ROC) analysis has also widely been used in medical data analysis to study the effect of varying the threshold on the numerical outcome of a diagnostic test. A ROC curve provides a graphical representation of the relationship between the true-positive and false-positive prediction rate of a model. The y-axis corresponds to the sensitivity of the model, i.e. how well the model is able to predict true positives. The x-axis corresponds to the specificity (expressed on the curve as 1-specificity), i.e. the ability of the model to identify true negatives. An increase in specificity (i.e. a decrease along the X-axis) results in an increase in sensitivity. The greater the sensitivity at high specificity values (i.e. high y-axis values at low X-axis values) the better the model. A numerical measure of the accuracy of the model can be obtained from the area under the curve (AUC), where an area of 1.0 signifies near perfect accuracy, while an area of less than 0.5 indicates that the model is worse than just random.

TABLE 2
Complement Factor H, short splice form: [N4411]

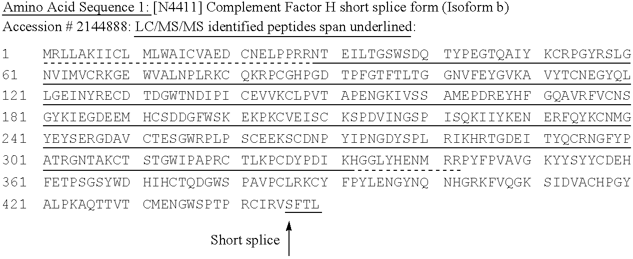

Alternative Names:
CFHL3; FHL1; HF1; HF2; HUS; MGC88246; beta-1-H-globulin
FACTOR H AND FACTOR H-LIKE 1, COMBINED DEFICIENCY OF
H factor 1 (complement)
H factor 2 (complement)
HF FACTOR H-LIKE 1
MACULAR DEGENERATION, AGE-RELATED, SUSCEPTIBILITY TO
complement factor H
complement factor H isoform a precursor
Complement factor H isoform b precursor
The gene sequence (variant 2) for Isoform b has multiple differences in the coding region, compared to variant 1. This includes an alternate Exon which results in an early stop Codon. The resulting protein (isoform b, also known as the “factor H-like 1” or “FHL-1” isoform) has a distinct C-terminus (arrow above) and is shorter than isoform (a) (139070 Da) shown below in Table 3):

TABLE 3
Amino Acid Sequence 2: Complement Factor H Isoform a, Long Splice Form:

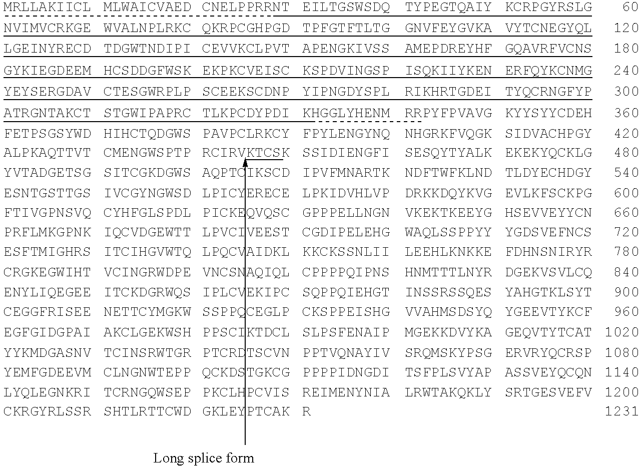

TABLE 4A

Amino acid Sequence 3; The 2D gel estimated pI = 6.3 and
MW = 39 KD. The best fit to these data for N4411:

N4411 LC MS/MS Peptide span + N terminal extension + HGGLYHENMRR
MRLLAKIICLMLWAICVAEDCNELPPRRNTEILTGSWSDQTYPEGTQAIYKCRPGYRSLGNVIMVCRKGEW
VALNPLRKCQKRPCGHPGDTPFGTFTLTGGNVFEYGVKAVYTCNEGYQLLGEINYRECDTDGWTNDIPICE
VVKCLPVTAPENGKIVSSAMEPDREYHFGQAVRFVCNSGYKIEGDEEMHCSDDGFWSKEKPKCVEISCKSP
DVINGSPISQKIIYKENERFQYKCNMGYEYSERGDAVCTESGWRPLPSCEEKSCDNPYIPNGDYSPLRIKH
RTGDEITYQCRNGFYPATRGNTAKCTSTGWIPAPRCTLKPCDYPDIKHGGLYHENMRR
pI: 6.3
MW: 38745

The presence of this identical sequence in both isoforms indicates that N4411 is derived from processing of either or both of the Isoforms (a and/or b). In order to find an amino acid sequence unique to either isoform a or b, also containing all the LC MS/MS peptide span of N4411, one must extend the carboxyl terminus beyond the splice junction, (for example below) which makes proteins that are much too large to fit the 2D gel data (Table 4B, C).

TABLE 4B

Amino acid sequence 4; next fit Total Factor H long splice
form amino acid Sequence 2 = N4411 LC MS/MS Peptide span +
C terminal extension:

NTEILTGSWSDQTYPEGTQAIYKCRPGYRSLGNVIMVCRKGEWVALNPLRKCQKRPCGHPGDTPFGTFTLT
GGNVFEYGVKAVYTCNEGYQLLGEINYRECDTDGWTNDIPICEVVKCLPVTAPENGKIVSSAMEPDREYHF
GQAVRFVCNSGYKIEGDEEMHCSDDGFWSKEKPKCVEISCKSPDVINGSPISQKIIYKENERFQYKCNMGY
EYSERGDAVCTESGWRPLPSCEEKSCDNPYIPNGDYSPLRIKHRTGDEITYQCRNGFYPATRGNTAKCTST
GWIPAPRCTLKPCDYPDIKHGGLYHENMRRPYFPVAVGKYYSYYCDEHFETPSGSYWDHIHCTQDGWSPAV
PCLRKCYFPYLENGYNQNHGRKFVQGKSIDVACHPGYALPKAQTTVTCMENGWSPTPRCIRVKTCS
pI: 6.9
MW: 47725
Too Large for N4411

TABLE 4C

Amino acid sequence 5: N4411 LC MS/MS Peptide span + C terminal
extension

NTEILTGSWSDQTYPEGTQAIYKCRPGYRSLGNVIMVCRKGEWVALNPLRKCQKRPCGHPGDTPFGTFTLT
GGNVFEYGVKAVYTCNEGYQLLGEINYRECDTDGWTNDIPICEVVKCLPVTAPENGKIVSSAMEPDREYHF
GQAVRFVCNSGYKIEGDEEMHCSDDGFWSKEKPKCVEISCKSPDVINGSPISQKIIYKENERFQYKCNMGY
EYSERGDAVCTESGWRPLPSCEEKSCDNPYIPNGDYSPLRIKHRTGDEITYQCRNGFYPATRGNTAKCTST
GWIPAPRCTLKPCDYPDIKHGGLYHENMRRPYFPVAVGKYYSYYCDEHFETPSGSYWDHIHCTQDGWSPAV
PCLRKCYFPYLENGYNQNHGRKFVQGKSIDVACHPGYALPKAQTTVTCMENGWSPTPRCIRVSFTL
pI: 6.6
MW: 47754
Too large for N4411

Thus, protein spot N4411 is a processing product derived from Complement Factor H Isoform a long splice form and/or Complement Factor H Isoform b short splice form.

Factor H promotes the release of Complement Factor Bb from the alternative C3 Convertase, inhibiting the Alternative Pathway of Complement activation. Low levels of Factor H result in increased activation of the alternative pathway, and are also associated with disorders involving micro-angiopathic vascular syndromes. Complement Factor H has been found localized in Amyloid (Abeta) plaques in Alzheimer's disease in the brain, as a function of microglial interaction (Strohemeyer et al. 2002, J Neuroimmunology 131: 135-146).

Protein spot N4411 in Normal Control Subjects, Patients Diagnosed with Parkinson's Disease, and Patients Diagnosed with PD-Like, and/or Mixed Disorders

The blood serum concentrations of a Complement Factor H processing product protein spot N4411 were determined in 57 normal controls, 29 Parkinson's disease patients (PD), 8 patients with Frontotemporal dementia (FTD), Lewy body dementia (LBD), Corticalbasal Ganglionic Degeneration, a form of Multiple System Atrophy (MSA I), 19 patients with Stroke-Related, Mixed and other PD-Like disorders, including another form of Multiple System Atrophy. As depicted in FIG. 2 and Table 5, The blood serum of patients with Frontotemporal dementia (FTD), Lewy body dementia (LBD), Corticalbasal Ganglionic Degeneration, and with a form of Multiple System Atrophy (MSA I), is characterized by significantly low concentrations of Complement Factor H processing product protein spot N4411, when compared to normal subjects and PD patients (ANOVA-P<0.0001). In addition, the patients with Stroke-Related, Mixed and other PD-Like disorders, including another form of Multiple System Atrophy are also characterized by significantly high blood serum concentrations of Complement Factor H processing product protein spot N4411, when compared to normal controls (ANOVA-P<0.0001). The patients with Stroke-Related, Mixed and other PD-Like disorders, included patients diagnosed with Multi-infarct dementia, CVA, Post-irradiation Encephalopathy and Seizures, Vascular Parkinsonism, Thalamic CVA and HX of Lung CA, Alzheimer's disease combined with Vascular (Multi-Infarct) dementia, Alzheimer's disease combined with Lewy body dementia, Parkinson's disease combined with Lewy body dementia, Alzheimer's and Parkinson's disease combined with Lewy body dementia, Frontotemporal dementia combined with Chronic Inflammatory Demyelinating Polyneuropathy, Alcohol related dementia, Idiopathic Sensory Ataxia, Semantic dementia, and patients with another form of Multiple System Atrophy (MSA II), were also characterized by significantly high blood serum concentrations of Complement Factor H processing product protein spot N4411, when compared to normal controls and patients with Lewy body dementia, Frontotemporal dementia, or a form of Multiple System Atrophy (MSA I) (ANOVA-P<0.0001).

As depicted in Table 5i, the mean level of blood serum concentrations of the Complement Factor H processing product protein spot N4411 in the group of 57 normal control individuals (100%) was 197.3±29.26 S.E. (PPM).

Also depicted in Table 5i, the mean level of blood serum concentrations of the Complement Factor H processing product protein spot N4411 in the group of 29 PD patients was 2.47 fold higher than normal controls (247%) at 486.7±50.97 S.E. (PPM).

Also depicted in Table 5i, the mean level of blood serum concentrations of the Complement Factor H processing product protein spot N4411 in the group of 8 patients with LBD, FTD, CBGD, and a form of Multiple System Atrophy (MSA I) was 0.6 fold lower than normal controls (40%) at 78.9±15.87 S.E. ppm.

Also depicted in Table 5i, the mean level of blood serum concentrations of the Complement Factor H processing product protein spot N4411 in the group of 19 patients with Stroke-Related, Mixed and other PD-Like disorders was 2.72 fold higher than normal controls (272%) at 535.6±42.42 S.E. ppm.

### Blood Serum Concentrations of the Complement Factor H Processing Product Protein Spot N4411 in the Diagnosis and Differential Diagnosis of Parkinson's Disease and Parkinson's Disease-Like Disorders

As depicted in Table 5i, the blood serum concentration values of Complement Factor H processing product protein spot N4411 for the population of the Parkinson's disease patients are substantially 2.5 fold higher than those of the normal control individuals, i.e. 6.2 fold higher than those of patients with Lewy Body dementia, Frontotemporal dementia, and a form of Multiple System Atrophy (MSA I). Also, the blood serum concentration values of Complement Factor H processing product protein spot N4411 of patients with Stroke-Related, Mixed (Alzheimer's disease and/or Parkinson's disease combined with Lewy Body Dementia or Vascular (Multi-Infarct) dementia, and/or those with Stroke-Related dementia) and other PD-Like disorders, including a form of Multiple System Atrophy (MSA II) were 6.8 fold higher than those with Lewy body dementia, Frontotemporal dementia, Corticalbasal Ganglionic Degeneration, or a form of Multiple System Atrophy (MSA I).

The differences in the blood serum concentrations of Complement Factor H processing product protein spot N4411 between the patient groups all display high degrees of statistical significance (P<0.0001) as shown in Table 5ii.

Hence, in one embodiment of the invention, the blood serum concentration of a Complement Factor H protein is used in the differential diagnosis of Parkinson's disease, and of Parkinson's disease Like disorders.

For the purpose of illustrating this preferred embodiment of the invention, the Receiver Operator Characteristics of blood serum concentrations of Complement Factor H protein N4411 were determined using Analyze-it software for Microsoft Excel, and the plots obtained are illustrated in FIGS. 3-6.

FIG. 3 illustrates the Receiver Operator Characteristics of blood serum concentrations of Complement Factor H protein N4411 in distinguishing between Parkinson's disease (PD) vs. PD-Like disorders: Lewy Body Dementia (LBD), Frontotemporal dementia (FTD), a form of Multiple System Atrophy (MSA I); and Cortical Basal Ganglionic Degeneration (CBGD). In this example, differential diagnosis of Parkinson's disease from PD-Like disorders: Lewy Body Dementia (LBD), Frontotemporal dementia (FTD), a form of Multiple System Atrophy (MSA I); and Cortical Basal Ganglionic Degeneration (CBGD) is demonstrated with an area of 0.811±SE 0.0425, P<0.0001. Using a cut off value of ≧166 PPM (Illustrated in FIG. 2) for Parkinson's disease, the assay produced a sensitivity of 70.1% for diagnosis of Parkinson's disease and a specificity of 95% for diagnosis of PD-Like disorders, including Lewy Body Dementia (LBD), Frontotemporal dementia (FTD), a form of Multiple System Atrophy (MSA I); and Cortical Basal Ganglionic Degeneration (CBGD).

This example demonstrates that the blood serum concentration of Complement Factor H protein N4411 has utility in differential diagnosis with a sensitivity of 70.1% of Parkinson's disease patients from Lewy Body Dementia (LBD), Frontotemporal dementia (FTD), a form of Multiple System Atrophy (MSA I); and Cortical Basal Ganglionic Degeneration (CBGD). The high specificity (95%) indicates that the assay is also useful in screening out patients with PD-Like disorders: Lewy Body Dementia (LBD), Frontotemporal dementia (FTD), a form of Multiple System Atrophy (MSA I); and Cortical Basal Ganglionic Degeneration (CBGD), for clinical trials or drug treatment, to treat Parkinson's disease patients separate from those with PD-Like disorders, including Lewy Body Dementia (LBD), Frontotemporal dementia (FTD), a form of Multiple System Atrophy (MSA I); and Cortical Basal Ganglionic Degeneration (CBGD).

FIG. 4 illustrates the Receiver Operator Characteristics of blood serum concentrations of Complement Factor H protein N4411 in distinguishing between PD-Like disorders, including Lewy Body Dementia (LBD), Frontotemporal dementia (FTD), a form of Multiple System Atrophy (MSA I); and Cortical Basal Ganglionic Degeneration (CBGD) vs. patients with Stroke-related, Mixed, or other non-Parkinson's neurological disorders, including another form of Multiple System Atrophy (MSA II). In this example, differential diagnosis of PD-Like disorders, including Lewy Body Dementia (LBD), Frontotemporal dementia (FTD), a form of Multiple System Atrophy (MSA I); and Cortical Basal Ganglionic Degeneration (CBGD) vs. patients with Stroke-related, Mixed, or other non-Parkinson's neurological disorders, including another form of Multiple System Atrophy (MSA II), is demonstrated with an area of 0.994±SE 0.0058, P<0.0001. Using a cut off value of ≧166 PPM (Illustrated in FIG. 2) for Lewy Body Dementia (LBD), Frontotemporal dementia (FTD), a form of Multiple System Atrophy (MSA I); and Cortical Basal Ganglionic Degeneration (CBGD), the assay produced a sensitivity of 90.0% for diagnosis of Lewy Body Dementia (LBD), Frontotemporal dementia (FTD), a form of Multiple System Atrophy (MSA I); and Cortical Basal Ganglionic Degeneration (CBGD) and a specificity of 98.0% for diagnosis of patients with Stroke-related, Mixed, or other non-Parkinson's neurological disorders, including another form of Multiple System Atrophy (MSA II).

This example demonstrates that the blood serum concentration of Complement Factor H protein N4411 has utility in differential diagnosis of 90.0% of patients with Lewy Body Dementia (LBD), Frontotemporal dementia (FTD), a form of Multiple System Atrophy (MSA I); and Cortical Basal Ganglionic Degeneration (CBGD) from patients with Stroke-related, Mixed, or other non-Parkinson's neurological disorders, including another form of Multiple System Atrophy (MSA II). The high specificity (98%) indicates that the assay is also useful in screening out patients with Stroke-related, Mixed, or other non-Parkinson's neurological disorders, including another form of Multiple System Atrophy (MSA II), for clinical trials or drug treatment, to treat patients with PD-Like disorders, including Lewy Body Dementia (LBD), Frontotemporal dementia (FTD), a form of Multiple System Atrophy (MSA I); and Cortical Basal Ganglionic Degeneration (CBGD) separate from those with Stroke-related, Mixed, or other non-Parkinson's neurological disorders, including another form of Multiple System Atrophy (MSA II).

FIG. 5 illustrates the Receiver Operator Characteristics of blood serum concentrations of Complement Factor H protein N4411 in distinguishing between Parkinson's disease (PD) vs. Normal Control subjects. In this example, differential diagnosis of Parkinson's disease from Normal Controls is demonstrated with an area of 0.728±SE 0.0370, P<0.0001. Using a cut off value of ≧166 PPM (Illustrated in FIG. 2) for Parkinson's disease, the assay produced sensitivity of 70.1% for diagnosis of Parkinson's disease and specificity of 64.1% for diagnosis of Normal Control individuals.

This example demonstrates that the blood serum concentration of Complement Factor H protein N4411 has utility in diagnosis of 70.1% of Parkinson's disease patients from Normal Control individuals.

FIG. 6 illustrates the Receiver Operator Characteristics of blood serum concentrations of Complement Factor H protein N4411 in distinguishing between patients with Stroke-related, Mixed, or other non-Parkinson's neurological disorders, including another form of Multiple System Atrophy (MSA II) vs. Normal Controls. In this example, diagnosis of PD-Like disorders, including Stroke-related, Mixed, or other non-Parkinson's neurological disorders, including another form of Multiple System Atrophy (MSA II), vs. Normal Controls is demonstrated with an area of 0.879±SE 0.0234, P<0.0001. Using a cut off value of ≧166 PPM (Illustrated in FIG. 2) for patients with Stroke-related, Mixed, or other non-Parkinson's neurological disorders, including another form of Multiple System Atrophy (MSA II), this assay demonstrated sensitivity of 98.0% and specificity for Normal Controls of 64.1%. Using a cut off value of ≧252 PPM for patients with Stroke-related, Mixed, or other non-Parkinson's neurological disorders, including another form of Multiple System Atrophy (MSA II) this assay demonstrated sensitivity of 91.8% and specificity for Normal Controls of 77.8%.

This example demonstrates that the blood serum concentration of Complement Factor H protein N4411 has utility in diagnosis of 90-98% of patients with Stroke-related, Mixed, or other non-Parkinson's neurological disorders, including another form of Multiple System Atrophy (MSA II) from Normal Controls.

The blood serum samples may also be subjected to various other techniques known in the art for separating and quantitating proteins. Such techniques include, but are not limited to: gel filtration chromatography, ion exchange chromatography, reverse phase chromatography, affinity chromatography (typically in an HPLC or FPLC apparatus), affinity capture, or any of the various centrifugation techniques well known in the art. Certain embodiments would also include a combination of one or more chromatography or centrifugation steps combined via electrospray or nanospray with mass spectrometry or tandem mass spectrometry of the proteins themselves, or of a total digest of the protein mixtures. Certain embodiments may also include surface enhanced laser desorption mass spectrometry or tandem mass spectrometry, or any protein separation technique that determines the pattern of proteins in the mixture, either as a one-dimensional, two-dimensional, three-dimensional or multi-dimensional protein pattern, and/or the pattern of protein post synthetic modifications or different isoforms of a Complement Factor H protein are used.

Quantitation of a protein by antibodies directed against that protein is well known in the field. The techniques and methodologies for the production of one or more antibodies to a Complement Factor H protein are routine in the field and are not described in detail herein.

As used herein, the term antibody is intended to refer broadly to any immunologic binding agent such as IgG, IgM, IgA IgD and IgE. Generally, IgG and/or IgM are preferred because they are the most common antibodies in the physiological situation and because they are most easily made in a laboratory setting.

Monoclonal antibodies (MAbs) are recognized to have certain advantages, e.g., reproducibility and large-scale production, and their use is generally preferred. The invention thus provides monoclonal antibodies of human, murine, monkey, rat, hamster, rabbit, chicken, or other animal origin. Due to the ease of preparation and ready availability of reagents, murine monoclonal antibodies are generally preferred. However, human auto antibodies or “humanized” antibodies are also contemplated, as are chimeric antibodies from mouse, rat, or other species, bearing human constant and/or variable region domains, bispecific antibodies, recombinant and engineered antibodies and fragments thereof.

The term “antibody” thus also refers to any antibody-like molecule that has a 20 amino acid antigen binding region, and includes antibody fragments such as Fab′, Fab, F(ab′)2, single domain antibodies (DABS), Fv, scFv (single chain Fv), and the like. The techniques for preparing and using various antibody-based constructs and fragments are well known in the art. Means of preparing and characterizing antibodies are also well known in the art (See, e.g., Antibodies: A Laboratory Manual, Cold Spring Harbor Laboratory, 1988; incorporated herein by reference).

Antibodies to a Complement Factor H protein may be used in a variety of assays in order to quantitate the protein in serum samples, or other fluid or tissue samples. Well known methods include immunoprecipitation, antibody sandwich assays, ELISA and affinity chromatography methods that include antibodies bound to a solid support. Such methods also include micro arrays of antibodies or proteins contained on a glass slide or a silicon chip, for example.

It is contemplated that arrays of antibodies to a Complement Factor H protein, or peptides derived from a Complement Factor H protein, may be produced in an array and contacted with the serum samples or protein fractions of serum samples in order to quantitate the blood serum concentrations of a Complement Factor H protein. The use of such micro arrays is well known in the art and is described, for example in U.S. Pat. No. 5,143,854, incorporated herein by reference.

The present invention includes a screening assay for neurodegenerative disease based on the up-regulation and/or down-regulation of a Complement Factor H protein expression. One embodiment of the assay will be constructed with antibodies to a Complement Factor H protein. One or more antibodies targeted to antigenic determinants of a Complement Factor H protein will be spotted onto a surface, such as a polyvinyl membrane or glass slide. As the antibodies used will each recognize an antigenic determinant of a Complement Factor H protein, incubation of the spots with patient samples will permit attachment of a Complement Factor H protein to the antibody.

The binding of a Complement Factor H protein can be reported using any of the known reporter techniques including radioimunoassays (RIA), stains, enzyme linked immunosorbant assays (ELISA), and sandwich ELISAs with a horseradish peroxidase (HRP)-conjugated second antibody also recognizing a Complement Factor H protein, the pre-binding of fluorescent dyes to the proteins in the sample, or biotinylating the proteins in the sample and using an HRP-bound streptavidin reporter. The HRP can be developed with a chemiluminescent, fluorescent, or colorimetric reporter. Other enzymes, such as luciferase or glucose oxidase, or any enzyme that can be used to develop light or color can be utilized at this step.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods, and in the steps or in the sequence of steps of the methods described herein without departing from the concept, spirit and scope of the invention.

More specifically, it is well recognized in the art that the statistical data, including but not limited to the mean, standard error, standard deviation, median, interquartile range, 95% confidence limits, results of analysis of variance, non-parametric median tests, discriminant analysis, Receiver Operator Characteristics (ROC), etc., will vary as data from additional patients are added to the database or antibodies are utilized to determine concentrations of a Complement Factor H protein or any biomarker. Therefore changes in the range of concentrations of a Complement Factor H protein do not depart from the concept, spirit and scope of the invention.

Also more specifically, it is disclosed (in cross referenced US Utility patent applications by Goldknopf, I. L. et al. Ser. Nos. 11/507,337 and 11/503,881, US Provisional Patent Applications by Goldknopf et al. Ser. No. 60/708,992 and 60/738,710, and referenced in Goldknopt I. L et al. 2006 and E. A. Sheta et al, 2006, hereby incorporated as reference) that blood serum concentrations of protein biomarkers, including Complement Factor H processing product protein spot N4411, can be used in combination with other biomarkers for diagnosis, differential diagnosis, and screening. Consequently, the use of Complement Factor H protein in conjunction with one or more additional biomarkers does not depart from the concept, spirit and scope of the invention.

It is also well recognized in the art that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

It is also well recognized in the art that there are other Non-Parkinson's disorders related to those already mentioned that are hereby included within the scope of the invention including but not limited to Alzheimer's disease, Atypical parkinsonism, Ataxia, Dystonia, Progressive Supranuclear Palsy, Essential tremor, Mild Cognitive Impairment, Amyotrophic Lateral Sclerosis, and any neurological disease or disorder, injury, depression or other psychiatric condition, or any other PD-Like disorder with symptoms similar to Parkinson's disease that results from any other cause.

TABLE 5
(i) Mean level (ppm) ± standard error (SE) of human Complement
Factor H processing product protein (spot N4411) in sera of PD
and PD-like disorders. (ii) Statistical significance (ANOVA-P) of
differences in serum concentrations

| | | Number | | % of |
|---|---|---|---|---|
| (i) | Classification | of Subjects | Mean ± SE | Control |
| | Control | 57 | 197.3 ± 29.26 | 100% |
| | PD | 29 | 486.7 ± 50.97 | 247% |
| | LBD, FTD, CBGD, MSA I | 8 | 78.9 ± 15.87 | 40% |
| | Stroke-Related, | 19 | 535.9 ± 42.42 | 272% |
| | Mixed, MSA II, | | | |
| | and other PD-Like | | | |
| | Disorders | | | |

| (ii) | Compared group | ANOVA-P |
|---|---|---|
| | Control vs. PD | <0.0001 |
| | Control vs. Stroke Related, Mixed, MSA II, | <0.0001 |
| | and other PD-Like Disorders | |
| | PD vs. LBD, FTD, CBGD, MSA I | <0.0001 |
| | LBD, FTD, CBGD, MSA I vs. Stroke Related, | <0.0001 |
| | Mixed, MSA II, and other PD-Like Disorders | |

What is claimed is:

1. A biomarker for diagnosis, differential diagnosis and screening for a neurodegenerative disease comprising a Complement Factor H protein in a blood serum sample.

2. The biomarker of claim 1, wherein the neurodegenerative disease is a form of movement disorder,

3. The biomarker of claim 1, wherein the neurodegenerative disease is Parkinson's disease.

4. The biomarker of claim 1, wherein the neurodegenerative disease is Frontotemporal dementia, Lewy body dementia, Corticalbasal Ganglionic Degeneration, or a form of Multiple System Atrophy.

5. The biomarker of claim 1, wherein the neurodegenerative disease is a Stroke-Related, disorder, including: Multi-Infarct or Vascular dementia, Cerebrovascular accident, Post-irradiation Encephalopathy with seizures, Vascular Parkinsonism, Thalamic Cerebrovascular accident.

6. The biomarker of claim 1, wherein the neurodegenerative disease is a Mixed disorder such as Alzheimer's, Parkinson's or both of Alzheimer's and Parkinson's, combined with vascular dementia, vascular parkinsonism, or Lewy body dementia, or Frontotemporal dementia combined with Chronic Inflammatory Demyelinating Polyneuropathy.

7. The biomarker of claim 1 wherein the neurodegenerative disease is Alcohol related dementia, Semantic dementia, Ataxia, Atypical parkinsonism, Dystonia, Progressive Supranuclear Palsy, Essential tremor, Alzheimer's disease, Mild Cognitive Impairment, Amyotrophic Lateral Sclerosis, and any neurological disease or disorder or injury, depression or other psychiatric condition, or any other Parkinson's disease-like with symptoms similar to Parkinson's disease that results from any other cause.

8. The biomarker of claim 1, wherein the Complement Factor H protein includes one or more of the amino acid sequences in Tables 2 and 3.

9. The biomarker of claim 1, wherein the Complement Factor H protein is one or more of the processing products included in the amino acid sequences in Tables 2, 3 and 4.

10. The biomarker of claim 1, wherein the Complement Factor H protein includes one or more antigenic determinants of the Complement Factor H protein, located within one or more of the amino acid sequences in Tables 2, 3 and 4.

11. The use of the biomarker of claim 1 in a method for screening, diagnosing and/or differentially diagnosing for a neurodegenerative disease comprising:
obtaining a blood, blood serum, or blood plasma sample from a test subject;
determining a quantity of a Complement Factor H protein in the subject sample; and
comparing the quantity of a Complement Factor H protein in the test subject sample with ranges of values of the quantity of a Complement Factor H protein in samples of normal control subjects; and one or more groups of patients with a neurodegenerative disease,
whereby a quantity of a Complement Factor H protein in the test subject sample is indicative of a neurodegenerative disease or a normal condition.

12. The method of claim 11, wherein the quantity of the biomarker in claim 1 is determined by two-dimensional gel electrophoresis.

13. The method of claim 11, wherein the two-dimensional gel electrophoresis comprises a separation by isoelectric point followed by a separation by molecular weight.

14. The method of claim 11, wherein the two-dimensional gel is stained and an intensity of the biomarker of claim 1 is proportional to the expression of the biomarker of claim 1 in the serum sample.

15. The method of claim 11, wherein the quantity of the biomarker in claim 1 is determined by one or more antibodies to one or more antigenic determinants of the Complement Factor H protein, located within one or more of the amino acid sequences in Tables 2, 3 and 4.

16. The method of claim 11, wherein the ranges of blood serum concentrations of a Complement Factor H protein in any group of normal controls or neurodegenerative diseases is determined by statistics.

17. The method of claim 11, wherein the quantity of a Complement Factor H protein is determined along with the quantity of one or more other biomarkers for diagnosis, differential diagnosis or screening for a neurodegenerative disease.

18. The method of claim 11, wherein the screening, diagnosis or differential diagnosis is an adjunct to at least one other diagnostic test for the neurodegenerative disease.

19. The method of claim 11, wherein the quantity of a Complement Factor H protein in the subject sample is determined by transferring the protein from the two-dimensional gel to a PVDF membrane (Western blot) and contacting the transferred protein with at least one antibody with reactivity to the amino acid sequences in Table 2, 3 and 4.

20. The method of claim 11, wherein the quantity of a Complement Factor H protein in the subject sample is determined by any type of immunoassay.
